(19) United States
(12) Patent Application Publication
(10) Pub. No.: US 2021/0145813 A1
(43) Pub. Date: May 20, 2021

(54) COMPOSITIONS AND METHODS FOR SUSTAINED RELEASE OF FLECAINIDE

(71) Applicant: ALSAR LTD PARTNERSHIP, Plantation, FL (US)

(72) Inventor: Robert FISHEL, Delray Beach, FL (US)

(73) Assignee: ALSAR LTD PARTNERSHIP, Plantation, FL (US)

(21) Appl. No.: 17/160,281

(22) Filed: Jan. 27, 2021

Related U.S. Application Data
(63) Continuation of application No. 17/047,662, filed on Oct. 14, 2020.
(60) Provisional application No. 62/657,947, filed on Apr. 16, 2018.

(51) Int. Cl. A61K 31/4458; A61K 9/48; A61K 31/138

(52) U.S. Cl. CPC A61K 31/4458; A61K 31/138; A61K 9/4808

(57) ABSTRACT

The invention relates generally to sustained release compositions. Specifically, the invention relates to biphasic and triphasic compositions and methods for controlling the release of a medication to treat a heart disease.

### CROSS-REFERENCE TO RELATED APPLICATIONS

This application claims priority to and the benefit of U.S. Provisional Patent Application 62/657,947, filed Apr. 16, 2018, which is incorporated by reference herein in its entirety.

### FIELD OF THE INVENTION

The invention relates generally to sustained release compositions. Specifically, the invention relates to biphasic and triphasic compositions and methods for controlling the release of a medication to treat a heart disease.

### BACKGROUND OF THE INVENTION

Many diseases require more than one medication for effective treatment and often require pre-treatment with a first medication followed by a second medication often times with both medications needed at prolonged active levels following dosing. In addition, some medications often need to be dosed two or more times daily and need to be combined with rate control agents during dosing to prevent contraindications. For diseases treated this way, multiple daily dosing with a rate control agent can cause both patient non-compliance as well as patient inconvenience.

In the field of heart disease, supraventricular tachycardia (herein abbreviated SVT), atrial fibrillation (herein abbreviated AFib) and atrial flutter (herein abbreviated AFL) are serious heart conditions treated by various medications having limited effectiveness for various reasons. For example, regarding AFib, the following medications are known as possible prescribed treatments: sotalol, dronaderone, dofetilide, propafenone, amiodarone and flecainide. Sotalol is associated with polymorphic sustained ventricular tachycardia (also known as torsades de pointes) and can cause sudden cardiac death even in patients with otherwise normal heartbeats. This medication requires hospital admission in order to initiate. Dronaderone can cause pulmonary fibrosis, hepatitis and can double the death rate in patients with heart failure. This medication produces fewer patient side effects yet is also less effective at treating AFib. Dofetilide carries a high risk of sudden cardiac death if started as an outpatient, requires special medical certification to prescribe and a three-day hospital stay to initiate. This medication may be more effective than others however it is also more dangerous and requires continuous lifelong monitoring once started to check for QT-interval prolongation. Propafenone carries less risk than other medications for treating AFib however requires multiple daily dosing, either as a BID (bis in die—twice daily) capsule with a sustained release formulation or as a TID (ter in die—thrice daily) capsule in a regular formulation. Amiodarone is known worldwide as a highly effective medication for treating AFib and can be administered as a once daily capsule however the medication is highly toxic and not approved for AFib treatment in the USA by the FDA. The medication causes thyroid abnormalities and can cause blindness, pulmonary fibrosis, hepatitis, anorexia and hypogonadism. Flecainide is also an effective medication for AFib as well as SVT however it must be taken as a BID capsule and needs to be combined with a rate control agent to prevent AV (atrioventricular) nodal conduction time increase should an episode of AFib occur.

In general, IC class anti-arrhythmic drugs, such as propafenone and flecainide cannot be administered alone for the treatment of many types of arrhythmias, including SVT, AFib and AFL, since such medications can paradoxically increase AV nodal conduction time while simultaneously slowing yet not terminating an underlying atrial arrhythmia. As an example, if a patient develops right atrial flutter (and more specifically tricuspid annular dependent right atrial reentry) then the heating rate in the right atrium may typically reach close to 300 heats per minute (herein abbreviated BPM). The electrical impulse arriving from the SA (sinoatrial) node to the atria needs to transit across the AV node in order to cause ventricular contraction. Normal AV nodal physiology prevents electrical conduction of an atrial beating rate as fast as 300 BPM. This is normal AV nodal physiology and can be considered a type of natural circuit breaker. A ventricular beating rate of 300 BPM is also too fast to allow for the mechanical contraction of the heart and typically would cause cardiac arrest and death. Given an episode of AFL, even though the atrium may beat at a rate of 300 BMP, the AV node will typically only conduct an electrical impulse every other beat or every third beat to the ventricles. During such an episode of AFL whereby the atrial beating rate is 300 BPM, the ventricular beating rate will be a fixed fraction of that rate, usually between 100-150 BPM. IC class anti-arrhythmic drugs can be used to prevent atrial arrhythmias such as AFL. However, prior to the termination of the tachycardia (fast beating of the heart), such drugs can both slow the rate of tachycardia in the atria and simultaneously increase the AV nodal conduction time. The effect of such actions is that a dosing of an anti-arrhythmic medication alone can cause the atrial beating rate to slow to around 200 BPM and yet by accelerating AV nodal conduction time, also enable a 1:1 ratio in atrial to ventricular conduction time thus producing a ventricular beating rate of 200 BPM and worsening the clinical status of the patient. To avoid such contraindications, these types of medications are given along with a rate control agent such as a beta blocker, a calcium channel blocker or digitalis, which acts to slow the AV nodal conduction time (also known as AV nodal blocking), thereby preventing the paradoxical increase in the ventricular beating rate and the potential worsening of the patient's condition. Typically rate control agents are given prior to the administration of IC class anti-arrhythmic medications so that a patient is protected from secondary rapid tachycardia caused by the increase in AV nodal conduction time.

As mentioned above, with the exception of the highly toxic amiodarone, IC class anti-arrhythmic medications often need to be dosed two or more times daily and need to be combined with rate control agents for each dosing thereby causing both inconvenience and non-compliance. Such methods of dosing are known in the art. An article entitled “Real-world safety and efficacy of a ‘pill-in-the-pocket’ approach for the management of paroxysmal atrial fibrillation” to Yao et al., published in the Canadian Journal of Cardiology, Volume 33, 2017, p.S190 is directed to a study on the treatment of AFib using an AV nodal blocker, such as diltiazem, verapamil or metoprolol 30 minutes prior to the administration of an oral dose of an IC class anti-arrhythmic drug, such as flecainide or propafenone. Treatment was first administered in an emergency room setting and was then transferred to out-of-hospital administration for patients meeting criteria of efficaciousness and treatment tolerance.

An article entitled “Flecainide-metoprolol combination reduces atrial fibrillation clinical recurrences and improves tolerability at 1-year follow-up in persistent symptomatic atrial fibrillation” to Capucci et al., published in Eurospace, Volume 18, 2016, pp. 1698-1704, is directed to a study on the efficacy and safety of a combination of flecainide and metoprolol in preventing AFib clinical recurrences. The study randomized patients into three groups, flecainide and metoprolol (group A), flecainide only (group B) and metoprolol only (group C). Groups A and B were given flecainide as a BID capsule, with group A given metoprolol also as a BID capsule. The flecainide and metoprolol combination therapy was found to improve effectiveness and increase tolerability.

Sustained release medications are also known in the art. U.S. Pat. No. 8,268,352 B2, to Vaya et al. and entitled “Modified release composition for highly soluble drugs” is directed to a modified release dosage form comprised of a high solubility active ingredient, which utilizes a dual retard technique to effectively reduce the quantity of release controlling agents.

The invention of Vaya et al. can also comprise another active ingredient as an immediate release form or a modified release form. The dosage form is comprised of micro matrix particles containing a high solubility active ingredient and one or more hydrophobic release controlling agents and a coating of micro matrix particles with one or more hydrophobic release controlling agents. The dosage form may also include one or more commonly used excipients in oral pharmaceutical formulations.

U.S. Pat. No. 9,554,989 B2 to Kaplan et al. and entitled “Silk reservoirs for drug delivery” is directed to silk-based drug delivery compositions that provide sustained delivery of therapeutic agents. In addition to fostering patient compliance, such silk-based drug delivery compositions exhibit excellent biocompatibility and non-inflammatory degradation products, such as peptides and amino acids. The silk compositions can be processed in completely aqueous based solvents. The silk-based drug delivery composition of Kaplan et al. comprises a therapeutic agent encapsulated in a substantially silk reservoir implant or silk injectable reservoir comprising silk fibroin. The ends of the silk reservoir implant or silk injectable reservoir are closed to form a silk reservoir implant or silk injectable reservoir. In addition, the silk-based drug delivery composition is capable of sustained delivery of the therapeutic agent in vivo. The invention of Kaplan et al. is also directed to a method of preparation comprising forming a silk tube from silk fibroin, loading the silk tube with a therapeutic agent and closing the silk tube ends such that the therapeutic agent is sealed therein. The closed tube ends can be coated with a polymer solution, such as a silk solution to form a silk reservoir implant or silk injectable reservoir. The silk tube for the silk reservoir implant or silk injectable reservoir can be made by gel-spinning in which the silk fibroin solution is delivered over a rotating mandrel which is simultaneously reciprocated horizontally. The silk fibroin forms a coating on the mandrel and the process can be repeated as many times as needed to obtain a desired number of coating layers or wall thickness for the silk reservoir implant or silk injectable reservoir.

Accordingly, there exists a need for an improved composition for controlling the release of flecainide.

### SUMMARY OF THE INVENTION

In one aspect, the invention provides a method for treating a heart disease, the method comprising administering to a subject in need thereof a composition comprising flecainide combined with a binding agent, wherein said binding agent is capable of facilitating a slow release of said flecainide over a predetermined time period for once daily dosing, and wherein said heart disease is supraventricular tachycardia, atrial fibrillation, atrial flutter, or a combination thereof.

In another aspect, the invention provides a composition for preventing AV nodal conduction time increase during treatment of atrial fibrillation, comprising: an IC class anti-arrhythmic drug; and a rate control agent. In an exemplary embodiment, said IC class anti-arrhythmic drug is flecainide acetate or flecainide tartrate. In another exemplary embodiment, said rate control agent is a beta blocker, a calcium channel blocker, a metoprolol, or a combination thereof.

In another aspect, the invention provides a biphasic delayed release capsule, comprising: a first compartment, containing a first medication; a second compartment, containing a second medication; a first coating, surrounding said first compartment; and a second coating, surrounding said second compartment and separating said first compartment from said second compartment, wherein said first coating dissolves immediately, thereby immediately releasing said first medication; and wherein said second coating is time released according to a predetermined time period, thereby providing a sustained release of said second medication.

In another aspect, the invention provides a triphasic delayed release capsule, comprising: a first compartment, containing a first medication; a second compartment, containing a second medication; a third compartment, containing a combination of said first and second medications; a first coating, surrounding said first compartment; a second coating, surrounding said second compartment and separating said first compartment from said second compartment; and a third coating, surrounding said third compartment and separating said second compartment from said third compartment, wherein said first coating dissolves immediately, thereby immediately releasing said first medication; wherein said second coating is time released according to a predetermined time period, thereby providing a sustained release of said second medication; and wherein said combination of said first and second medications is combined with a binding agent for sustained release of said combination of said first and second medications over a controlled release time period.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will be understood and appreciated more fully from the following detailed description taken in conjunction with the drawings in which:

FIG. 1 is a schematic illustration of a biphasic delayed release capsule, constructed and operative in accordance with an embodiment of the invention;

FIG. 2 is a schematic illustration of a triphasic delayed release capsule, constructed and operative in accordance with another embodiment of the invention;

FIG. 3 is a graph showing relative flecainide levels as a function of time using the triphasic delayed release capsule of FIG. 2, constructed and operative in accordance with a further embodiment of the invention; and

FIG. 4 is a graph showing relative drug concentration as a function of time using the triphasic delayed release capsule of FIG. 2, constructed and operative in accordance with another embodiment of the invention.

### DETAILED DESCRIPTION OF THE INVENTION

The invention overcomes the disadvantages of the prior art by providing a system and method for the delivery of two or more medications in a sequential and then controlled and/or delayed manner for the treatment of disease in a once daily format. The compositions and methods of the invention can be used, for example, in the treatment of heart diseases such as SVT, AFib and AFL, where an initial concentration level of a first medication may be required before the delivery of a second medication which should be released in the body in a controlled manner. According to the invention, the treatment of diseases which require more than one medication dosed more than once a day can be delivered to a patient in a once daily format using a single daily pill, thereby increasing patient compliance and convenience. Whereas the invention is primarily described using the example of the treatment of heart diseases such as SVT, AFib and AFL with an IC class anti-arrhythmic drug combined with a rate control agent, the invention can be used to treat other diseases which require treatments that involve multiple medications dosed more than once a day.

As mentioned above in the background section, IC class anti-arrhythmic drugs cannot be given alone for the treatment of diseases such as SVT, AFib and AFL since such drugs can paradoxically increase AV nodal conduction time while simultaneously slowing yet not terminating an underlying atrial arrhythmia. In order to prevent the increase in AV nodal conduction time (also known as AV nodal blocking), IC class anti-arrhythmic drugs are given with a rate control agent such as a beta blocker, a calcium channel blocker or digitalis to keep AV nodal conduction time from increasing. According to one aspect of the invention, a drug delivery system and method of preparation is provided wherein an IC class anti-arrhythmic drug is combined with a rate control agent, thus enabling both drugs to be delivered effectively and appropriately at therapeutic levels over the course of a 24-hour period in a single pill. Throughout the description, the terms “medication”, “drug” and “agent” are used interchangeably to describe a compound having therapeutic capabilities. In addition, the terms “pill”, “capsule” and “tablet” are used interchangeably to describe a physical structure containing therein a medication or drug which can be ingested or swallowed. Furthermore, the terms “sustained release”, “controlled release” and “delayed release” are used interchangeably to describe the release of a medication in the body of a patient over a predetermined amount of time.

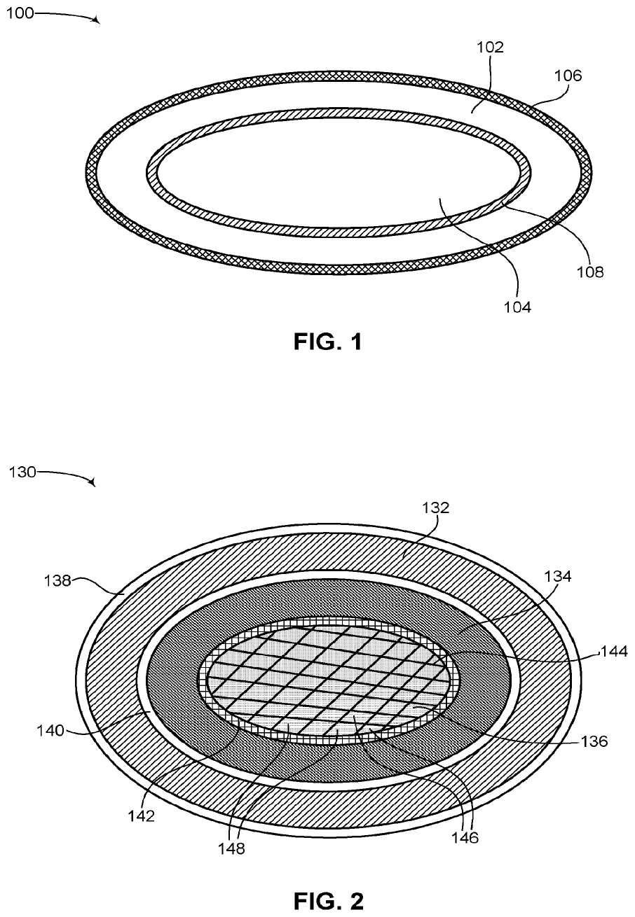

Reference is now made to FIG. 1, which is a schematic illustration of a biphasic delayed release capsule, generally referenced 100, constructed and operative in accordance with an embodiment of the disclosed technique. Biphasic delayed release capsule 100 includes a first compartment 102 and a second compartment 104. Each one of compartments 102 and 104 can house a drug or medication of a given dosage. First compartment 102 is surrounded by a first coating 106 and second compartment 104 is surrounded by a second coating 108. Second coating 108 separates first compartment 102 from second compartment 104. First coating 106 and second coating 108 may be time released such that they dissolve in the digestive system only after a predetermined amount of time, such as 30 minutes, 60 minutes, 3 hours, 6 hours and the like. According to the disclosed technique, before an IC class anti-arrhythmic drug (herein abbreviated ICAA drug) is delivered to a patient to treat SVT, AFib or AFL, a rate control agent should be delivered and should be in high enough concentration in the body before the ICAA drug is delivered. According to the disclosed technique, a rate control agent, such as a beta blocker, a calcium channel blocker, or a digitalis, is placed in first compartment 102. An ICAA drug is placed in second compartment 104. The ICAA drug may be flecainide acetate, flecainide tartrate or propafenone. The ICAA drug may be mixed with a binding agent to allow for slow release of the ICAA drug over a 6-24 hour period.

First coating 106 may be a dummy coating which dissolves almost immediately (for example within a few seconds to within a few minutes in the digestive system) whereas second coating 108 may be time released to dissolve within 1-2 hours. In the case of heart diseases such as SVT, AFib and AFL, biphasic delayed release capsule 100 as described above would enable an initial delivery of a rate control agent into the digestive system and then the blood stream of a patient followed by the release of an ICAA drug after 1-2 hours, thereby preventing the ICAA drug from increasing the AV nodal conduction time due to the presence of the rate control agent. As mentioned above, the ICAA drug may be combined with a binding agent for enabling sustained release of the ICAA drug over the course of 6-24 hours. A patient thus could take biphasic delayed release capsule 100 once in the morning, knowing that the ICAA drug would be released over the course of the day and ensuing night along with the rate control agent being present in the body of the patient and preventing an increase in AV nodal conduction time.

Reference is now made to FIG. 2, which is a schematic illustration of a triphasic delayed release capsule, generally referenced 130, constructed and operative in accordance with another embodiment of the disclosed technique. Triphasic delayed release capsule 130 includes three compartments, a first compartment 132, a second compartment 134 and a third compartment 136. First compartment 132 is surrounded by a first coating 138. Second compartment 134 is surrounded by a second coating 140 which separates first compartment 132 from second compartment 134. Third compartment 136 is surrounded by a third coating 142 which separates second compartment 134 from third compartment 136. Each one of first, second and third coatings may be timed released coatings or may dissolve almost immediately (within seconds to minutes upon ingestion). As shown, third compartment 136 includes a matrix or mesh 144, schematically representing a combination of at least two drugs or medications which are either chemically mixed together or are physically bonded together, for example via a binding agent, and are released in a sustained manner as the matrix or mesh dissolves.

According to another aspect of the invention, triphasic delayed release capsule 130 can be used to treat patients suffering from a disease in which treatment requires an initial boost of a first medication, followed by the release of a second medication and then followed by a sustained release of the first and second medication together. One such example may be the treatment of heart diseases such as SVT, AFib and AFL using a rate control agent (first medication) and an ICAA drug (second medication). As shown, a first medication may be placed in first compartment 132 and a second medication may be placed in second compartment 134. First coating 138 may be a dummy coating which dissolves within seconds and/or minutes in the digestive system, thereby immediately releasing the first medication into the blood stream of the patient. Second coating 140 may also be a dummy coating or may be time released after a predetermined amount of time, such as 30 minutes, 60 minutes or 2-3 hours. After second coating 140 dissolves, the second medication in second compartment 134 is released into the blood stream of the patient. Third coating 142 may also be time released or may be a dummy coating. Matrix 144 holds together a mixture of the first medication and the second medication, shown schematically as sections 146 and 148, with each section representing a different medication. Sections 146 and 148 may be held together using a binding agent (not shown) which slowly dissolves in the digestive system, thereby slowly releasing the first and second medications into the body. Sections 146 and 148 may be held together by a binding agent such as a polymer matrix or a clay matrix. The binding agent may be matrix 144. The first and second medications may also be chemically combined, depending on their respective compositions, in matrix 144 and released in a sustained manner in the blood stream once third coating 142 and matrix 144 dissolve.

According to another aspect of the invention, triphasic delayed release capsule 130 can be used to treat patients suffering from SVT, AFib and/or AFL wherein a multiple dosing of an ICAA drug and a rate control agent are required throughout a 24-hour period. For example, the ICAA drug may be flecainide acetate or flecainide tartrate and the rate control agent may be metoprolol succinate. In this example, first compartment 132 contains the rate control agent and first coating 138 is a dummy coating. Second compartment 134 contains the ICAA drug and second coating 140 is a time released coating. Third compartment 136 contains a mixture of the ICAA drug as well as the rate control agent and third coating 142 is also a time released coating. Once swallowed, first coating 138 is immediately dissolved and the rate control agent is absorbed into the digestive system of the patient at a therapeutic concentration level. This prevents the increase in the AV nodal conduction time of the heart of the patient. After a delay based on the time release of second coating 140, the ICAA drug is released into the digestive system of the patient. The delay may be 3-6 hours. The ICAA drug and the rate control agent provide treatment to the patient for SVT, AFib and AFL for a number of hours. After another 3-6 hours, third coating 142 dissolves and the mixture of the ICAA drug and the rate control agent in matrix 144 is allowed to dissolve in the digestive system of the patient. Matrix 144 may include a clay or polymer mixture allowing for a slow and timed release of both the ICAA drug and the rate control agent, thereby allowing these two drugs to slowly absorb into the blood stream of the patient over the next 6-18 hours. Using a single pill (i.e., triphasic delayed release capsule 130), according to the disclosed technique, a patient thereby has sufficient medication, both an ICAA drug and a rate control agent, released over a period of approximately 24 hours, to effectively and properly treat SVT, AFib and/or AFL. Such a pill, according to the disclosed technique, could also be used as an emergency measure to treat a sudden episode of AFib. It is noted that according to the disclosed technique, third compartment 136 could include other medications to achieve other therapeutic effects, depending on the medical needs of the patient. For example, third compartment 136 could also include an anticoagulant drug, a blood thinning drug and the like.

According to another aspect of the invention, triphasic delayed release capsule 130 allows for the sequential controlled delivery of both a rate control agent and an ICAA drug as well as a delayed delivery of both drugs, thereby eliminating the need for BID or TID dosing of those drugs, thus increasing patient compliance and convenience in the treatment of heart disease. The disclosed technique has been described above using the example of flecainide and metoprolol in the treatment of heart diseases such as SVT, AFib and AFL, however the disclosed technique can be used for the treatment of other diseases requiring a similar treatment of at least two medications wherein an initial serum boost of a first medication is required, followed by a delivery of a second medication and then followed by a controlled release of a combination of both the first medication and the second medication.

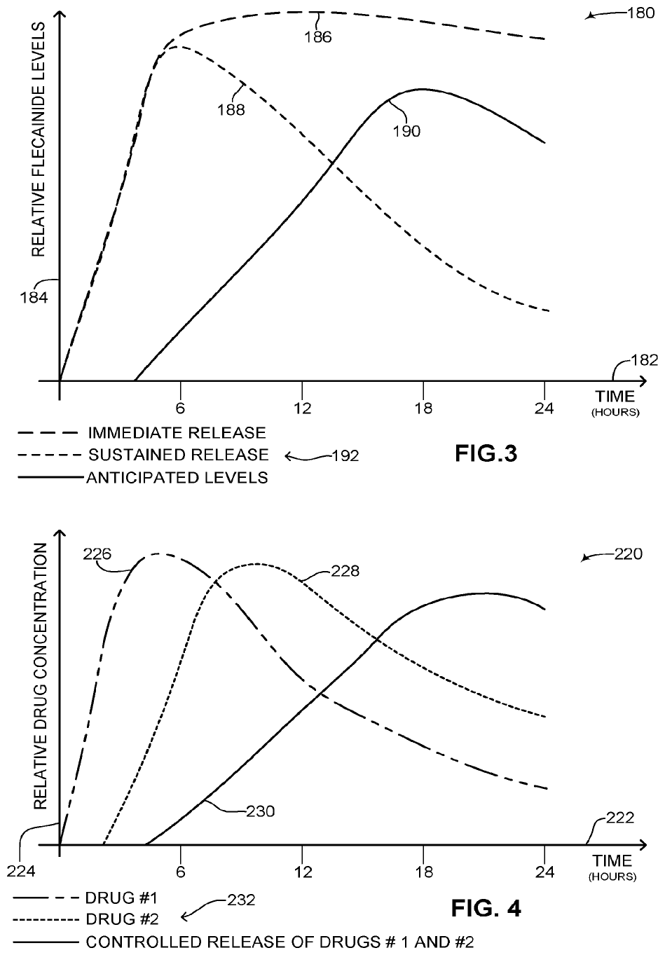

Reference is now made to FIG. 3, which is a graph showing relative flecainide levels as a function of time using the triphasic delayed release capsule of FIG. 2, generally referenced 180, constructed and operative in accordance with a further embodiment of the disclosed technique. Graph 180 includes an X-axis 182 showing time in hours and a Y-axis 184 showing relative flecainide levels (no units). Y-axis 184 can also represent relative concentration levels of any ICAA drug. A first curve 186 shows the relative concentration levels of flecainide released from the second compartment of triphasic delayed release capsule 130 (FIG. 2), a second curve 188 shows the relative concentration levels of flecainide released from the third compartment of triphasic delayed release capsule 130 and a third curve 190 show the anticipated overall concentration levels of flecainide in the blood stream based on the release of flecainide from both the second and third compartments of triphasic delayed release capsule 130. A legend 192 shows that first curve 186 represents the immediate release of flecainide without any sustained release whereas second curve 188 represents the release of flecainide with a sustained and controlled release over time. Third curve 190 represents the anticipated levels of flecainide in the body over time based on an immediate release of flecainide followed by a sustained release of flecainide. As shown in FIG. 3, the first dosing of flecainide from the second compartment, as shown by first curve 186, peaks after about 6 hours once all the medication has been absorbed from the digestive system into the blood stream, and slowly begins to lower over the course of the next 18-hour period. The second dosing of flecainide from the third compartment, as shown by second curve 188, also peaks close to 6 hours after ingestion, however since this dosing is either combined with a binding agent or a slow release agent, the amount of flecainide present in the blood stream quickly diminishes over the course of the next 18-hour period, as small amounts of flecainide are released from the third compartment over the course of the timed release, usually between 12-18 hours. Third curve 190 shows that the anticipated actual amount of available flecainide in the blood stream steadily increases from around 4 hours after ingestion of the pill until about 18 hours when the total amount begins to decline. As shown, triphasic delayed release capsule 130 allows for therapeutic levels of flecainide to be in the blood stream of a patient for a time period of substantially 24-hours, thus enabling effective treatment against SVT, AFib and AFL in a once daily pill.

Reference is now made to FIG. 4, which is a graph showing relative drug concentration as a function of time using the triphasic delayed release capsule of FIG. 2, generally referenced 220, constructed and operative in accordance with another embodiment of the disclosed technique. Graph 220 includes an X-axis 222 showing time in hours and a Y-axis 224 showing relative drug concentration levels (no units). A first curve 226 shows the relative concentration levels of a first medication released from the first compartment of triphasic delayed release capsule 130 (FIG. 2), a second curve 228 shows the relative concentration levels of a second medication released from the second compartment of triphasic delayed release capsule 130 and a third curve 230 show the relative concentration levels of a controlled release of the first medication and the second medication from the third compartment of triphasic delayed release capsule 130. A legend 232 shows that first curve 226 represents the release of a first medication after the dissolution of a first coating, second curve 228 represents the release of a second medication after the dissolution of a second coating and third curve 230 represents the release of a combination of the first and second medication in a timed release, for example if the first and second medications are combined using a binding agent, after the dissolution of a third coating.

The first medication could be a rate control agent, such as metoprolol whereas the second medication could be an ICAA drug, such as flecainide. As shown in FIG. 4, once the first coating of triphasic delayed release capsule 130 is dissolved, the first medication is absorbed into the blood stream, peaking in concentration at around 4-5 hours after ingestion. The second coating of triphasic delayed release capsule 130 may have a timed release, thereby only releasing the second medication contained in the second compartment starting around 3 hours after ingestion. As shown in FIG. 4, second medication is absorbed into the blood stream after a controlled delay, peaking in concentration at around 8-10 hours after ingestion. The third coating of triphasic delayed release capsule 130 may also have a timed release, thereby only releasing the combination of the first and second medication in the third compartment starting around 5 hours after ingestion. As shown in FIG. 4, since the combination of the first and second medications in the third compartment may be combined with a binding agent or polymer matrix, the release of the two medications together has a sustained release and thus the relative concentration levels of the two medications slowly rises and peaks around after 20 hours of ingestion. The peak levels of first, second and third curves 226, 228 and 230 represent therapeutic levels of both the first and second medications. As shown, using the delayed released system of triphasic delayed release capsule 130, therapeutic levels of the first medication and the second medication can be achieved over the course of a 24-hour period thus enabling a single once daily pill to provide therapeutic drug levels to a patient for treating a variety of diseases, including but not limited to heart diseases such as SVT, AFib and AFL. In the case of SVT, Afib or AFL, the first medication, as shown by first curve 226 is immediately released, thus serving the function of an AV nodal blocker. The second medication, as shown by second curve 228, is only released a few hours later, thereby serving the function of treating an arrhythmia such as SVT, AFib or AFL once an AV nodal blocker is already in the patient's blood stream. The combination of the first and second medications, as shown by third curve 230, is finally released and begins to circulate in the blood stream once the initial dosing of the first and second medications begins to wear off (around 12 hours after initial ingestion). Since the combination of the first medication and the second medication is a timed released combination, both medications will remain in the blood stream until a 24-hour period from initial ingestion has passed at which point a patient ingests another once daily pill.

In a preferred embodiment, ICAA drug of the invention is a flecainide.

In another embodiment, a rate control agent of the invention is a beta blocker, a calcium channel blocker, or a digitalis.

### Beta Blockers

Beta blockers (also referred as β-blockers or beta blocker drugs) are a class of medications that are predominantly used to manage abnormal heart rhythms, and to protect the heart from a second heart attack (myocardial infarction) after a first heart attack (secondary prevention). They are also widely used to treat high blood pressure (hypertension).

Beta blockers are competitive antagonists that block the receptor sites for the endogenous catecholamines epinephrine (adrenaline) and norepinephrine (noradrenaline) on adrenergic beta receptors, of the sympathetic nervous system.

Some block activation of all types of β-adrenergic receptors and others are selective for one of the three known types of beta receptors, designated β₁, β₂ and β₃ receptors. β₁-adrenergic receptors are located mainly in the heart and in the kidneys. β₂-adrenergic receptors are located mainly in the lungs, gastrointestinal tract, liver, uterus, vascular smooth muscle, and skeletal muscle. β₃-adrenergic receptors are located in fat cells.

In one embodiment, the beta blocker drug of the invention is a non-specific or non-selective beta blocker drug.

In another embodiment, the beta blocker drug of the invention is a specific or selective beta blocker drug. In one example, the beta blocker drug of the invention specifically or selectively blocks the activation of β₁ receptor.

In another example, the beta blocker drug of the invention specifically or selectively blocks the activation of β₂ receptor. In yet another example, the beta blocker drug of the invention specifically or selectively blocks the activation of β₃ receptor.

Examples of a non-specific or non-selective beta blocker drug include, for example, but not limited to propranolol, bucindolol, carteolol, carvedilol, labetalol, nadolol, oxprenolol, penbutolol, pindolol, sotalol, and timolol.

Examples of β₁-selective or β₁-specific beta blockers include, for example, but not limited to, acebutolol, atenolol, betaxolol, bisoprolol, celiprolol, metoprolol, nebivolol, and esmolol.

β₁-selective or β₁-specific beta blockers are also known as cardioselective beta blockers. In a preferred embodiment, the beta blocker drug is a β₁-selective or β₁-specific beta blocker.

Examples of β₂-selective or β₂-specific beta blockers include, for example, but not limited to, butaxamine and ICI-118,551.

Examples of β₃-selective or β₃-specific beta blockers include, for example, but not limited to, SR 59230A.

In one embodiment, the beta blocker drug is a β₁ selective antagonist and β₃ agonist agent. Example of such β₁ selective antagonist and β₃ agonist agent includes, but not limited to, nebivolol.

Other examples of a beta blocker drug include, but not limited to, bisoprolol, metoprolol, nadolol, betaxolol, bisoprolol, esmolol, alprenolol, bucindolol, levobunolol, medroxalol, mepindolol, metipranolol, propafenone (propafenone is a sodium channel blocking drug that also is a beta-adrenergic receptor antagonist), propranolol, sotalol, and timolol.

### Calcium Channel Blocker

Calcium channel blockers are well known in the art and fully described in U.S. Pat. Nos. 10,117,848; 9,132,200; 8,748,648; 8,318,721; 5,209,933; and 4,552,881, and U.S. Patent Application Publications 20150335628; 20140323529; and 20110098273, which are incorporated by reference herein in their entirety.

Calcium channel blockers (CCB) are medications that disrupt the movement of calcium (Ca²⁺) through calcium channels. Calcium channel blockers are particularly effective against large vessel stiffness, one of the common causes of elevated systolic blood pressure in elderly patients. Calcium channel blockers are also frequently used to alter heart rate, to prevent cerebral vasospasm, and to reduce chest pain caused by angina pectoris.

N-type, L-type, and T-type voltage-dependent calcium channels are present in the zona glomerulosa of the human adrenal gland, and calcium channel blockers can directly influence the biosynthesis of aldosterone in adrenocortical cells, with consequent impact on the clinical treatment of hypertension with these agents.

In one embodiment, calcium channel blockers are dihydropyridine (DHP) calcium channel blockers. Examples of dihydropyridine (DHP) calcium channel blockers include, for example, but not limited to, amlodipine (Norvasc), aranidipine (Sapresta), azelnidipine (Calblock), barnidipine (HypoCa), benidipine (Coniel), cilnidipine (Atelec, Cinalong, Siscard), clevidipine (Cleviprex), efonidipine (Landel), felodipine (Plendil), isradipine (DynaCirc, Prescal), lacidipine (Motens, Lacipil), lercanidipine (Zanidip), manidipine (Calslot, Madipine), Nicardipine (Cardene, Carden SR), nifedipine (Procardia, Adalat), nilvadipine (Nivadil), nimodipine (Nimotop), nisoldipine (Baymycard, Sular, Syscor), nitrendipine (Cardif, Nitrepin, Baylotensin), and pranidipine (Acalas).

In another embodiment, calcium channel blockers are non-dihydropyridine calcium channel blockers. Examples of non-dihydropyridine calcium channel blockers include, for example, but not limited to, phenylalkylamine and benzothiazepine. Examples of phenylalkylamine include, for example, but not limited to verapamil (Calan, Isoptin), fendiline, and gallopamil. Examples of benzothiazepine include, for example, but not limited to, diltiazem (Cardizem).

In some embodiments, calcium channel blockers are nonselective, which include, for example, but not limited to mibefradil, bepridil, flunarizine, fluspirilene, and fendiline.

Other examples of calcium channel blockers include, for example, but not limited to, Ziconotide peptide and Gabapentinoids, such as gabapentin and pregabalin.

In a particular embodiment, calcium channel blockers are, for example, dihydropyridines (e.g. amlodipine), benzothiapines (e.g. diltiazem), and phenylalkylamines (e.g. verapamil), felodipine, nifedipine.

### Digitalis

In a particular embodiment, the digitalis is a digitalis glycoside. Examples of a digitalis glycoside include, for example, but not limited to oleandrin, neriifolin, odoroside A and H, ouabain (G-strophantin), cymarin, sarmentocymarin, periplocymarin, K-strophantin, thevetin A, cerberin, peruvoside, thevetosin, thevetin B, tanghinin, deacetyltanghinin, echujin, hongheloside G, honghelin, periplocin, strophantidol, nigrescin, uzarin, calotropin, cheiroside A, cheirotoxin, euonoside, euobioside, euomonoside, lancetoxin A and B, kalanchoside, bryotoxin A-C, bryophyllin B, cotiledoside, tyledoside A-D, F and G, orbicuside A-C, alloglaucotoxin, corotoxin, coroglaucin, glaucorin, scillarene A and B, scilliroside, scilliacinoside, scilliglaucoside, scilliglaucosidin, scillirosidin, scillirubrosidin, scillirubroside, proscillaridin A, rubelin, convalloside, convallatoxin, bovoside A, glucobovoside A, bovoruboside, antiarin A, helleborin, hellebrin, adonidin, adonin, adonitoxin, thesiuside, digitoxin, gitoxin, gitalin, digoxin, F-gitonin, digitonin, lanatoside A-C, bufotalin, bufotalinin, bufotalidin, pseudobufotalin, acetyl-digitoxin, acetyl-oleandrin, beta-methyldigoxin, and alpha-methyldigoxin.

In another particular embodiment, the digitalis glycoside is digitoxin or digoxin.

It will be appreciated by persons skilled in the art that the disclosed technique is not limited to what has been particularly shown and described hereinabove. Rather the scope of the disclosed technique is defined only by the claims, which follow.

The compositions described herein can be used to treat any suitable mammal, including primates, such as monkeys and humans, horses, cows, cats, dogs, rabbits, and rodents such as rats and mice. In one embodiment, the mammal to be treated is human.

All patents and literature references cited in the present specification are hereby incorporated by reference in their entirety.

The following examples are provided to supplement the prior disclosure and to provide a better understanding of the subject matter described herein. These examples should not be considered to limit the described subject matter. It is understood that the examples and embodiments described herein are for illustrative purposes only and that various modifications or changes in light thereof will be apparent to persons skilled in the art and are to be included within, and can be made without departing from, the true scope of the invention.

### EXAMPLES

### Example 1

The following formulation method is an example of the preparation of a biphasic delayed release capsule having flecainide 150 mg. The capsule has a core compartment (i.e., inner compartment) and an outer compartment.

The core compartment includes flecainide 150 mg coated with a polymer and the outer compartment includes metoprolol 50 mg (i.e., a rate control agent) coated with a polymer.

### Example 2

The following formulation method is an example of the preparation of a triphasic delayed release capsule having flecainide 150 mg. The capsule has a core compartment (i.e., inner compartment), a middle compartment, and an outer compartment.

The core compartment includes flecainide 150 mg mixed with metoprolol 50 mg (i.e., a rate control agent) coated with a polymer, the mixture coated with a polymer, the middle compartment includes flecainide 150 mg coated with a polymer, and the outer compartment includes metoprolol 50 mg (i.e., a rate control agent) coated with a polymer.

### Example 3

The formulation described in Example 1 or 2 can be orally administered to a subject.

Serum can be collected and analyzed. The flecainide composition may achieve a therapeutic effect within 2 hrs and maintain therapeutic effect for at least 24 hours in >95% percent of treated patients.

The composition may allow for consistent release of the active agent from the drug delivery vehicle with no more than 25% variation plus an encapsulation efficiency of over 70%. The composition may release the active agent from the drug delivery vehicle with >85% intact over the entire duration of release.

Having described preferred embodiments of the invention, it is to be understood that the invention is not limited to the precise embodiments, and that various changes and modifications may be effected therein by those skilled in the art without departing from the scope or spirit of the invention as defined in the appended claims.

1.-11. (canceled)

12. A biphasic delayed release capsule, comprising:
a first compartment, containing a rate control agent;
a second compartment, containing an IC class anti-arrhythmic drug;
a first coating, surrounding said first compartment; and
a second coating, surrounding said second compartment and separating said first compartment from said second compartment,
wherein said first coating dissolves immediately, thereby immediately releasing said rate control agent;
wherein said second coating is time released according to a predetermined time period, thereby providing a sustained release of said IC class anti-arrhythmic drug; and
wherein said rate control agent is metoprolol and wherein said IC class anti-arrhythmic drug is flecainide.

13. The biphasic delayed release capsule according to claim 12, wherein said IC class anti-arrhythmic drug is selected from the group consisting of flecainide acetate and flecainide tartrate.

14. The biphasic delayed release capsule according to claim 12, wherein said predetermined time period is 6-24 hours.

15. A triphasic delayed release capsule, comprising:
a first compartment, containing a rate control agent;
a second compartment, containing an IC class anti-arrhythmic drug;
a third compartment, containing a combination of the rate control agent and the IC class anti-arrhythmic drug;
a first coating, surrounding said first compartment;
a second coating, surrounding said second compartment and separating said first compartment from said second compartment; and
a third coating, surrounding said third compartment and separating said second compartment from said third compartment,
wherein said first coating dissolves immediately, thereby immediately releasing said rate control agent;
wherein said second coating is time released according to a predetermined time period, thereby providing a sustained release of said IC class anti-arrhythmic drug;
wherein said combination of the rate control agent and the IC class anti-arrhythmic drug is combined with a binding agent for sustained release of said combination of the rate control agent and the IC class anti-arrhythmic drug over a controlled release time period; and
wherein said rate control agent is metoprolol and wherein said IC class anti-arrhythmic drug is flecainide.

16. The triphasic delayed release capsule according to claim 15, wherein said IC class anti-arrhythmic drug is selected from the group consisting of flecainide acetate and flecainide tartrate.

17. The triphasic delayed release capsule according to claim 15, wherein said predetermined time period is between 3-6 hours and said controlled release time period is between 6-18 hours.

18. A method of treating supraventricular tachycardia, atrial fibrillation, and/or atrial flutter, the method comprising orally administering once daily to a subject in need thereof the biphasic delayed release capsule according to claim 12.

19. A method of treating supraventricular tachycardia, atrial fibrillation, and/or atrial flutter, the method comprising orally administering once daily to a subject in need thereof the triphasic delayed release capsule according to claim 15.
